(19) United States
(12) Patent Application Publication
(10) Pub. No.: US 2010/0022891 A1
(43) Pub. Date: Jan. 28, 2010

(54) INTRALUMINAL SPECTROSCOPE WITH WALL CONTACTING PROBE

(75) Inventors: Andres ZULUAGA, Boston, MA (US); Simon Furnish, New York, NY (US); Jay Caplan, Belmont, MA (US)

Correspondence Address: ROTHWELL, FIGG, ERNST & MANBECK, P.C., 1425 K STREET, N.W., SUITE 800, WASHINGTON, DC, 20005, US

(73) Assignee: INFRAREDX, INC., Cambridge, MA (US)

(21) Appl. No.: 12/568,431

(22) Filed: Sep. 28, 2009

Related U.S. Application Data
(62) Division of application No. 10/940,468, filed on Sep. 14, 2004.
Application No. 10/940,468 is a continuation of application No. PCT/US04/19883, filed on Jun. 21, 2004.
Application No. PCT/US04/19883 is a continuation of application No. 10/602,345, filed on Jun. 23, 2003, now abandoned.

(51) Int. Cl. A61B 5/00

(52) U.S. Cl. 600/473; 600/476; 600/478; 600/479

(57) ABSTRACT

A spectroscope for detecting vulnerable plaque within a lumen defined by an intraluminal wall includes a probe through which an optical fiber extends. An coupler in optical communication with the fiber is configured to atraumatically contact the intraluminal wall. A light source provides light to the fiber for illuminating the wall and a detector coupled to the fiber receives light from within the wall.

### FIELD OF INVENTION

The invention relates to spectroscopy, and in particular, to spectroscopes for detecting vulnerable plaques within a wall of a blood vessel.

### BACKGROUND

Atherosclerosis is a vascular disease characterized by a modification of the walls of blood-carrying vessels. Such modifications, when they occur at discrete locations or pockets of diseased vessels, are referred to as plaques. Certain types of plaques are associated with acute events such as stroke or myocardial infarction. These plaques are referred to as “vulnerable plaques.” A vulnerable plaque typically includes a lipid-containing pool of necrotic debris separated from the blood by a thin fibrous cap. In response to elevated intraluminal pressure or vasospasm, the fibrous cap can become disrupted, exposing the contents of the plaque to the flowing blood. The resulting thrombus can lead to ischemia or to the shedding of emboli.

One method of locating vulnerable plaque is to peer through the arterial wall with infrared light. To do so, one inserts a catheter through the lumen of the artery. The catheter includes a delivery fiber for illuminating a spot on the arterial wall with infrared light. Various particles in the blood, as well as the arterial wall itself, scatter or reflect much of this light. A small portion of the light, however, penetrates the arterial wall, scatters off structures deep within the wall. Some of this deeply-scattered light re-enters the lumen. This re-entrant light can be collected by a collection fiber within the catheter and subjected to spectroscopic analysis.

In an effort to avoid recovering light scattered from the blood and from the wall surface, the delivery fiber is displaced from the collection fiber. The diameter of the catheter must therefore be large enough to accommodate the two fibers and the gap that separates them.

### SUMMARY

The invention is based on the recognition that by collecting scattered light directly from an intraluminal wall, one avoids scattering that results from propagation of light through blood. As a result, it is no longer necessary to provide separate collection and delivery fibers. Instead, only a single fiber is necessary.

In one aspect, the invention includes a spectroscope for detecting vulnerable plaque within a lumen defined by an intraluminal wall. The spectroscope includes a probe having one or more optical fiber extending therethrough, and an atraumatic coupler in communication with the optical fiber(s). The coupler is configured to atraumatically contact the intraluminal wall. The spectroscope also includes a light source in optical communication with the fiber for illuminating the wall; and a detector in optical communication with the fiber for detecting light from within the wall.

In one embodiment, the probe includes a jacket enclosing the fiber. The jacket can be a coil-wire wound into a coil-wire jacket, with or without a variable diameter coil wire.

In other embodiments, the probe resiliently assumes a preferred shape. Examples of preferred shapes include a bow, an arc, a catenary, or a portion thereof.

The atraumatic coupler can be on the distal end of the probe. Embodiments of this type include those in which the atraumatic coupler is a lens attached to the distal tip of the optical fiber. Additional embodiments include those in which the atraumatic coupler is integral with the optical fiber, as for example where a distal tip of the optical fiber forms part of the atraumatic coupler.

The atraumatic coupler can also be along a side of the probe. Examples of such couplers include those having a window along a side of the probe, and a beam re-director providing optical communication between the window and a distal tip of the fiber. Other examples include those in which a distal face of the optical fiber provides optical communication with the window.

The invention optionally includes a cannula through which the probe passes. The cannula can include walls forming a channel conformal with the cannula through which the probe passes. In these embodiments, the probe can be steered toward the wall by providing tapered or flared distal end having an opening facing toward or away from a longitudinal axis of the cannula.

Other embodiments include those having a hub to which a distal end of the probe is attached, and those in which a cannula is provided for the hub and probe to pass through. In these embodiments, the probe can be one that resiliently assumes a bow shape for contacting the intraluminal wall at a point of inflection thereof. A coupler can then be placed at the point of inflection.

In another aspect, the invention includes a spectroscope having a cannula and a plurality of probes extending through the cannula. Each probe has an optical fiber extending therethrough, and an atraumatic coupler in communication with the optical fiber. The coupler is configured to atraumatically contact the intraluminal wall.

Some embodiments include a spacer ring attached to each of the probes for maintaining the positions of the probes relative to each other. Others include a hub attached to a distal end of each of the probes.

Another aspect of the invention is a method of detecting vulnerable plaque within an intraluminal wall. The method includes placing an atraumatic light coupler in contact with the intraluminal wall and passing light through the intraluminal wall by way of the atraumatic light coupler. Light from within the intraluminal wall is then recovered by way of the atraumatic coupler. This light is then provided to a processor for analysis to identify the presence of a vulnerable plaque.

In some practices of the method, placing an atraumatic light coupler in contact with the intraluminal wall includes placing a distal end of a probe in contact with the intraluminal wall. In other practices of the invention, it is a side of the probe that is placed in contact with the intraluminal wall.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE FIGURES

FIG. 1 is a schematic diagram of a spectroscope for identifying vulnerable plaque.

FIG. 2 is a schematic view of a probe in contact with the arterial wall.

FIG. 3 is a cross-section of the probe of FIG. 2.

FIGS. 4A-G are exemplary atraumatic light-couplers for an optical fiber.

FIGS. 5A-F are schematic views of single-probe spectroscopes.

FIGS. 6A-F are schematic views of multi-probe spectroscopes.

FIG. 7A is a schematic view of a probe emerging from a cannula having a tapered distal end.

FIG. 7B is a schematic view of a probe emerging from a cannula having a flared distal end.

FIGS. 8A-8F are schematic views of multi-probe spectroscopes in which the atraumatic light-couplers are along the sides of the probes.

FIGS. 8G-K are schematic views of spectroscopes in which the probes are integrated into the cannula.

FIGS. 9A-D are views of exemplary atraumatic light-couplers for the probes in FIGS. 8A-H.

### DETAILED DESCRIPTION

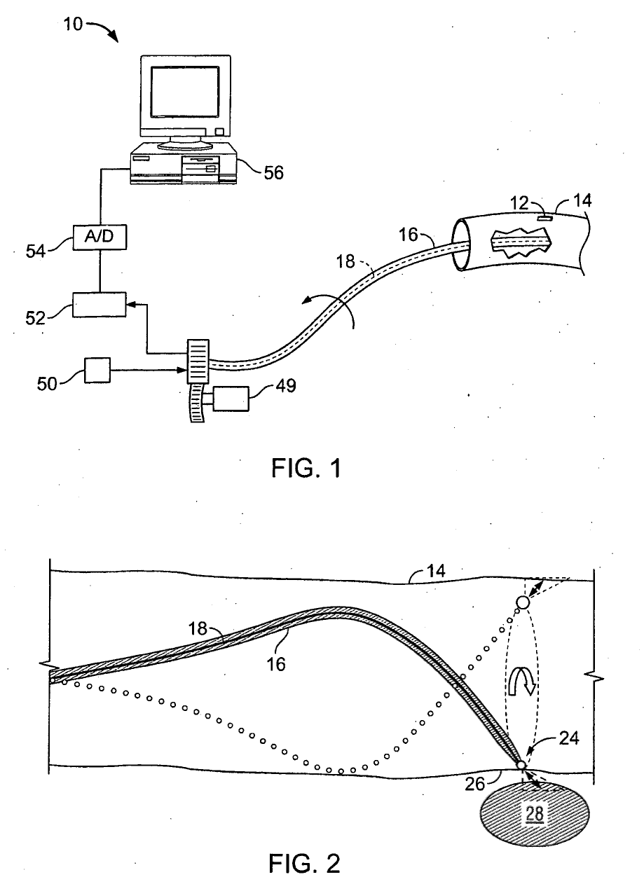

FIG. 1 shows a spectroscope 10 for identifying vulnerable plaque 12 in an arterial wall 14 of a patient. The spectroscope features a probe 16 to be inserted into a selected artery, e.g. a coronary artery, of the patient. An optical fiber 18 extends between a distal end and a proximal end of the probe 16.

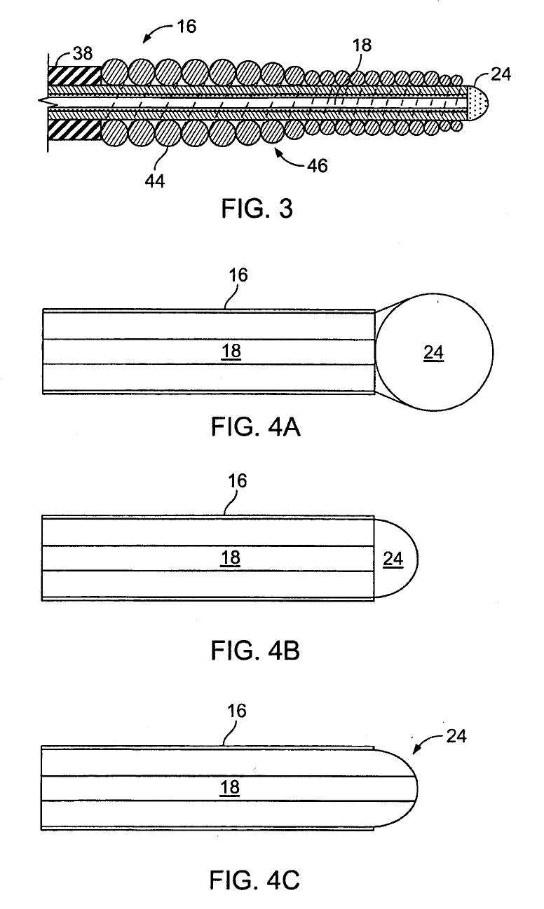

In a first embodiment, shown in FIGS. 2-3, an atraumatic light-coupler 24 at the distal end of the probe 16 rests on a contact area 26 on the arterial wall 14. When disposed as shown in FIG. 2, the atraumatic light-coupler 24 directs light traveling axially on the fiber 18 to the contact area 26. After leaving the atraumatic light-coupler 24, this light crosses the arterial wall 14 and illuminates structures 28 behind the wall 14. These structures 28 scatter some of the light back to the contact area 26, where it re-emerges through the arterial wall 14. The atraumatic light-coupler 24 collects this re-emergent light and directs it into the fiber 18.

Along a proximal section of the probe 16, as shown in FIG. 3, a rigid tube 38 encasing the fiber 18, enables the probe 16 to be pushed through the artery. Along a central and distal section of the probe 16, a coil wire 44 wound into a flexible coil-wire jacket 46 encases the fiber 18.

The coil wire 44 has a constant diameter along the central section. Along the distal section of the probe 16, the diameter of the coil wire 44 becomes progressively smaller. As a result, the distal section of the probe 16 is more flexible than its central section. This enhanced flexibility enables the distal section to follow the contour of the wall 14 without exerting unnecessary force against it.

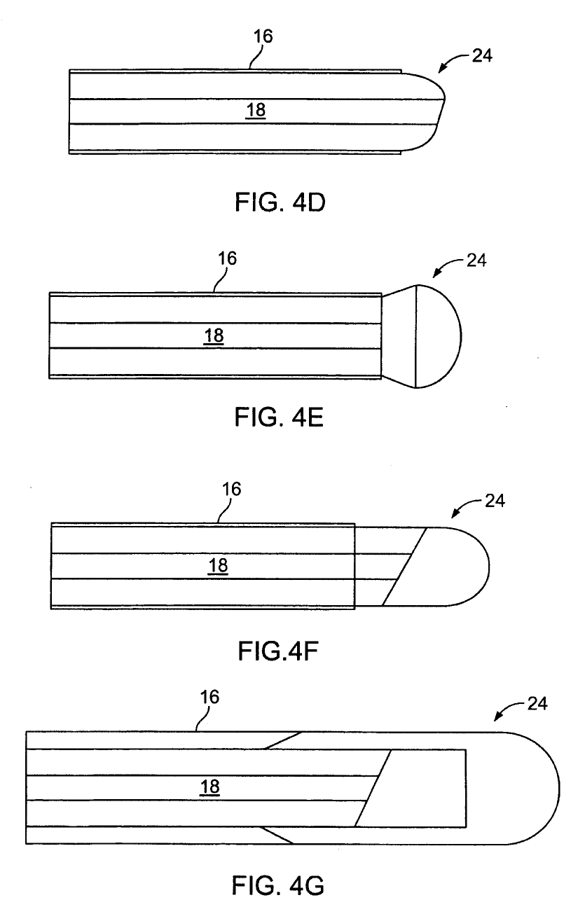

The atraumatic light-coupler 24 can be formed by attaching a lens assembly to a distal tip of the fiber 18, as shown in FIGS. 4A, 4B, and 4E, or by attaching a rounded glass tip to an angled fiber, as shown in FIGS. 4F-G. Alternatively, the atraumatic light-coupler 24 can be made integral with the fiber 18 by smoothing any sharp edges at its distal tip, as shown in FIGS. 4C-D.

In either case, the atraumatic light-coupler 24 can include a spherical lens, as shown in FIG. 4A, or a hemispherical lens, as shown in FIG. 4B. The atraumatic light-coupler 24 can also include more than one lens element, as shown in FIG. 4E.

Alternatively, the atraumatic light-coupler 24 can be integral with the fiber 18. For example, the distal tip of the fiber 18 can be formed into a plane having rounded edges and oriented at an angle relative to the plane of the fiber cross-section, as shown in FIG. 4D, or into a hemisphere, as shown in FIG. 4C.

Referring back to FIG. 1, one using the spectroscope 10 positions the atraumatic light-coupler 24 against the arterial wall 14 and engages a motor 49 coupled to the probe 16. The motor 49 rotates the probe 16 at a rate between approximately 1 revolution per second and 400 revolutions per second. This causes the atraumatic light-coupler 24 to trace a path around the inner circumference of the arterial wall 14. As it rotates, the atraumatic light coupler 24 redirects light placed on the fiber 18 by a light source 50, such as a near infrared light source, to the contact area 26. At the same time, the atraumatic light-coupler 24 collects light re-emerging from the contact area 26 and directs it into the fiber 18, which then guides it to a photo-detector 52.

The photo-detector 52 provides an electrical signal indicative of light intensity to an analog-to-digital (“A/D”) converter 54. The A/D converter 54 converts this signal into digital data that can be analyzed by a processor 56 to identify the presence of vulnerable plaque hidden beneath the arterial wall 14.

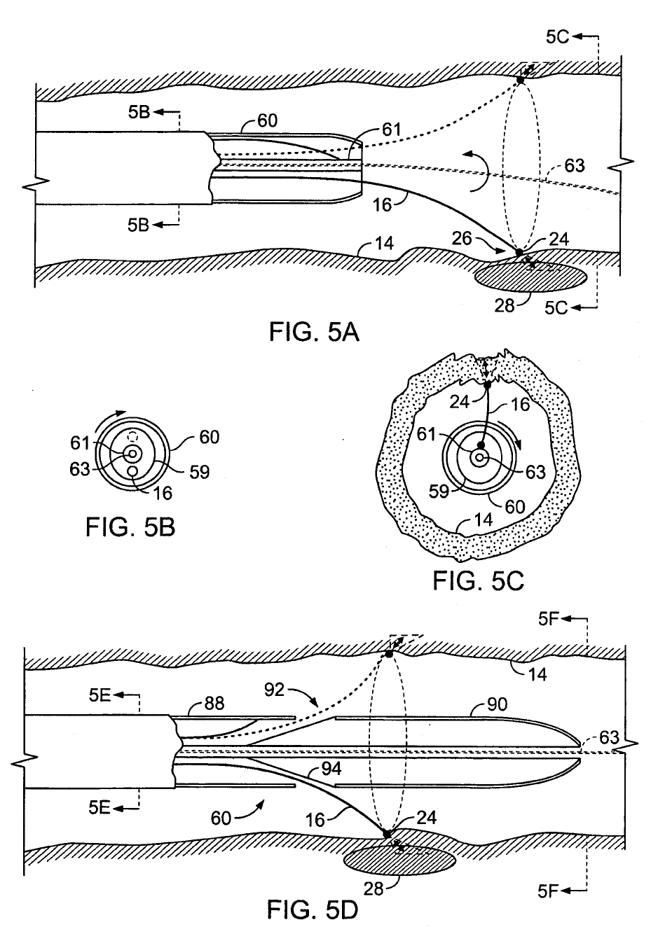

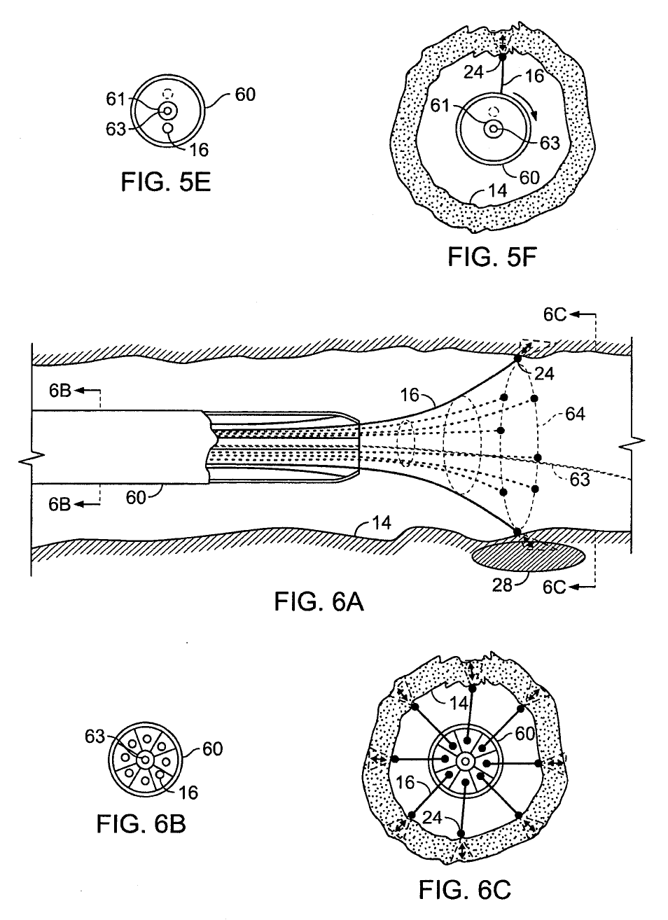

In a second embodiment, shown in FIGS. 5A-C, a probe housing 59 extends through a cannula 60 parallel to, but radially displaced from a longitudinal axis thereof. A probe 16 is kept inside the probe housing 59 until it is ready to be deployed. Extending along the longitudinal axis of the cannula 60 is a guide-wire housing 61 forming a guide-wire lumen through which a guide-wire 63 extends.

The probe 16 can be an optical fiber made of glass or plastic, or a bundle of such fibers. In one embodiment, the probe includes a bundle of 25 optical fibers, each 0.005 millimeters in diameter. The fiber(s) can be exposed, coated with a protective biocompatible layer and/or a lubricious layer such as polytetrafluoroethylene (“PTFE”), or encased in a coil-wire jacket. The optional coating or jacket around the fiber(s) could be round, and hence bendable in all directions, or flat, so as to suppress bending in undesired directions.

The distal tip of the optical fiber 18 is capped by any of the atraumatic light-couplers 24 discussed above. When the distal end of the cannula 60 is just proximal to contact area 26, the probe 16 is pushed distally so that its distal tip extends past the distal end of the cannula 60. Alternatively, the probe 16 remains stationary while the cannula 60 is retracted, thereby exposing the probe 16.

The probe 16 is pre-formed so that a natural bend urges it outward, away from the axis of the cannula 60. As a result, when the probe 16 is extended out its housing 59 and beyond the distal end of the cannula 60, this natural bend places the atraumatic light-coupler 24 of the fiber 18 in contact with the arterial wall 14 distal to the cannula 60. The probe 16 is then rotated so that the atraumatic light-coupler 24 traces, out a circular contact path along an inner circumference of the wall 14, as shown in FIGS. 5A and 5C.

A variety of ways are known for pre-forming a probe 16. For example, the probe 16 can be heated while in the desired shape. Or a coating over the fiber within the probe 16 can be applied and cured while the fiber is in the desired shape.

In a third embodiment, shown in FIGS. 5D-F, the cannula 60 has a proximal section 88 and a distal section 90 separated from each other by a circumferential gap 92. A guide wall 94 forms a truncated cone extending distally from a truncated end joined to the guide-wire housing 59 to a base joined to the distal section 90 of the cannula 60. The guide wall 94 thus serves to maintain the position of the proximal and distal sections 88, 90 of the cannula 60 relative to each other while preserving the circumferential gap 92 all the way around the cannula 60.

In use, the probe 16 is extended distally toward the guide wall 94, which then guides the probe 16 out of the circumferential gap 62. As was the case with the second embodiment (FIGS. 5A-C), the natural bend of the probe 16 urges the atraumatic tip 24 into contact with the arterial wall 14. Once the probe's atraumatic tip 24 contacts the wall 14, the probe 16 is rotated as shown in FIGS. 5D-F so that the atraumatic tip 24 sweeps a circumferential contact path on the arterial wall 14.

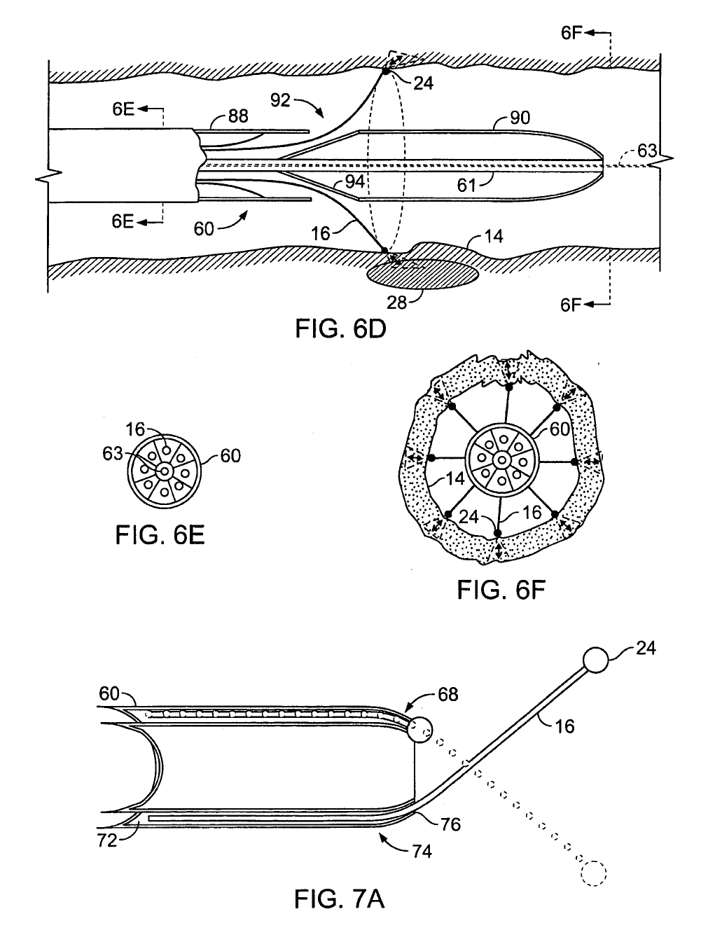

In a fourth embodiment, shown in FIGS. 6A-C, several probes 16 of the type discussed above in connection with FIGS. 5A-F pass through the cannula 60 at the same time. Optional spacer rings 64 are attached to the probes 62 at one or more points along their distal sections. The spacer rings 64 can be silicon webbing, plastic, Nitinol, or any other biocompatible material.

When deployed, the spacer rings 64 are oriented so as to lie in a plane perpendicular to the longitudinal axis of the cannula 60. The spacer rings 64 thus maintain the relative positions of the probes 16 during scanning of the wall 14. A multi-probe embodiment as shown in FIGS. 6A-C enables most of the circumference of an arterial wall 14 to be examined without having to rotate the probes 16.

In a fifth embodiment, shown in FIGS. 6D-F, the cannula 60 is as described in connection with the third embodiment (FIGS. 5D-F). The difference between this fifth embodiment and the third embodiment (FIGS. 5D-F) is that in the third embodiment, a single probe 16 extends through the circumferential gap 92, whereas in this fifth embodiment, several probes 16 circumferentially offset from one another extend through the circumferential gap 92. As a result, in the third embodiment, it is necessary to rotate the probe 16 to inspect the entire circumference of the arterial wall 14, whereas in the fifth embodiment, one can inspect most of the arterial wall 14 circumference without having to rotate the probes 16 at all.

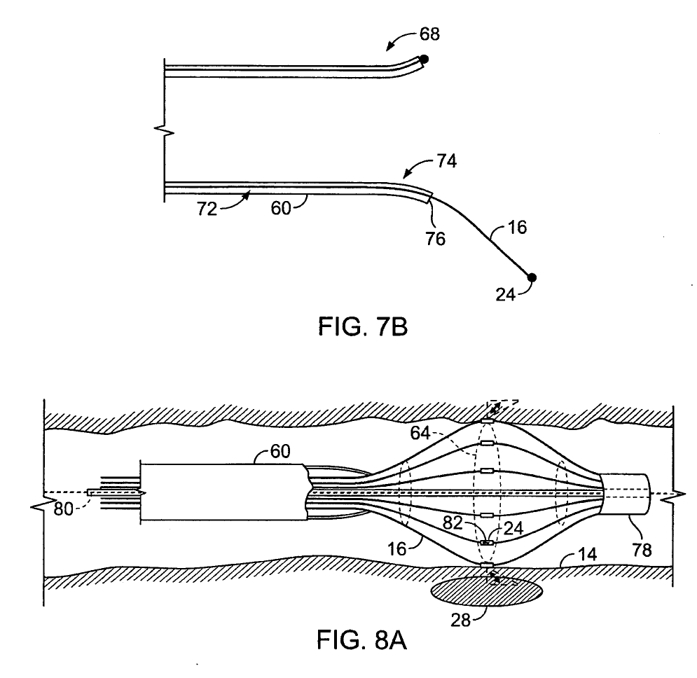

In a sixth embodiment, a cannula 60 has a tapered distal end 68, as shown in FIG. 7A, or a flared distal end 70, as shown in FIG. 7B. A channel 72 formed in the inner wall of the cannula 60 has a bend 74 proximal to an opening 76 at the distal end. This opening 76 defines a surface whose normal vector has both a radial component and an longitudinal component.

One operating the embodiments of FIGS. 7A and 7B pushes the probe 16 through the channel 72, which then guides it toward the opening 72. As the probe 16 exits the channel 72, it proceeds in the direction of the normal vector until its atraumatic light-coupler 24 contacts the arterial wall 14. In this case, the probe 16 need not be pre-formed to have a preferred shape since the channel 72 guides the probe 16 in the correct direction for reaching the wall 14.

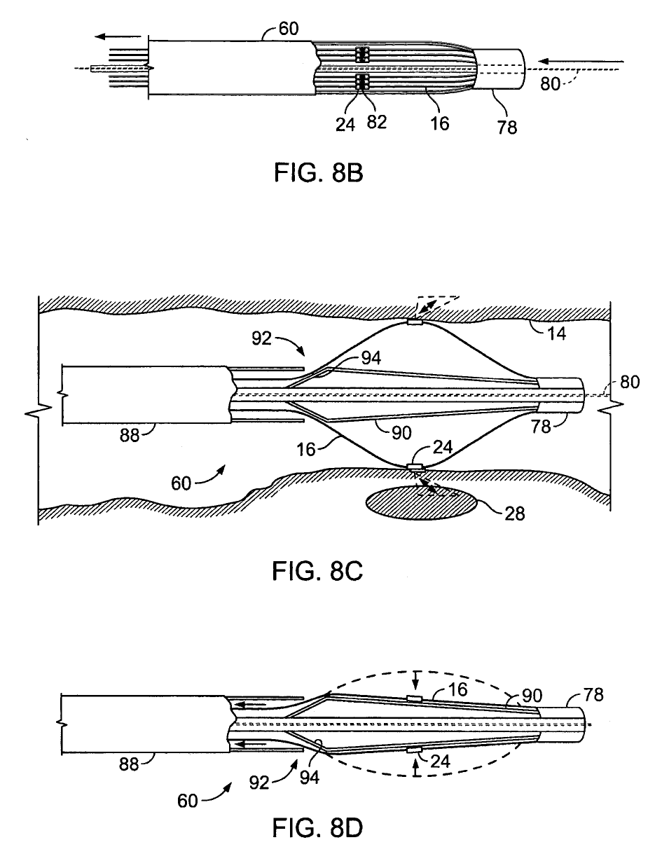

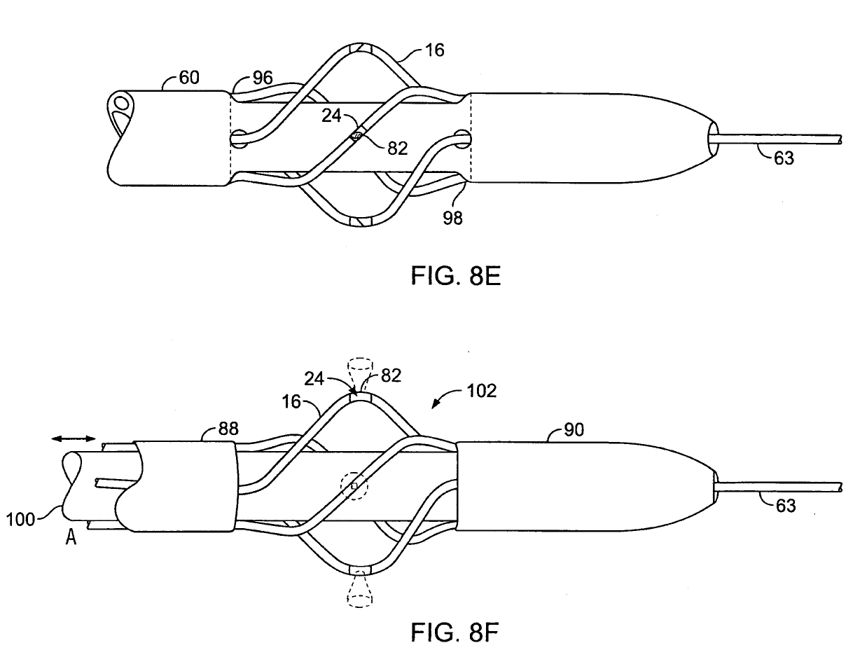

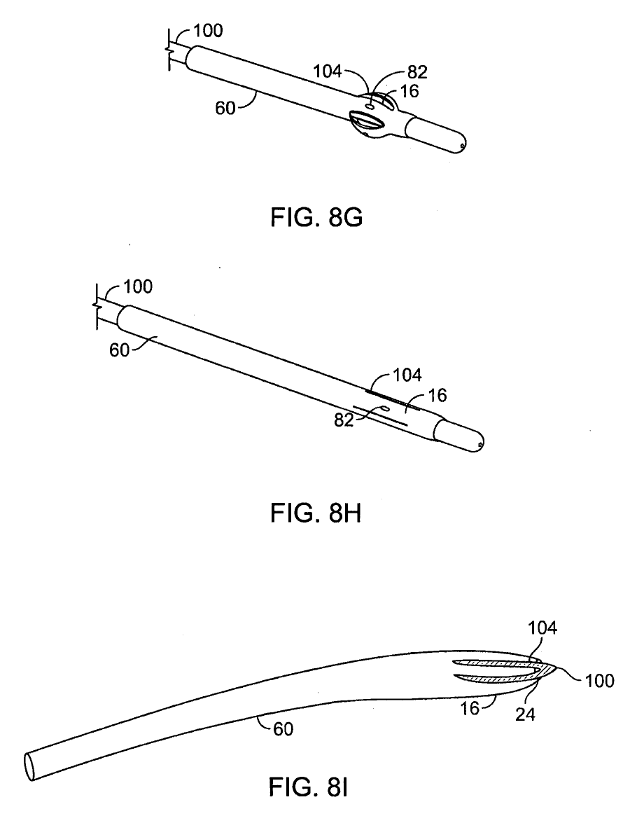

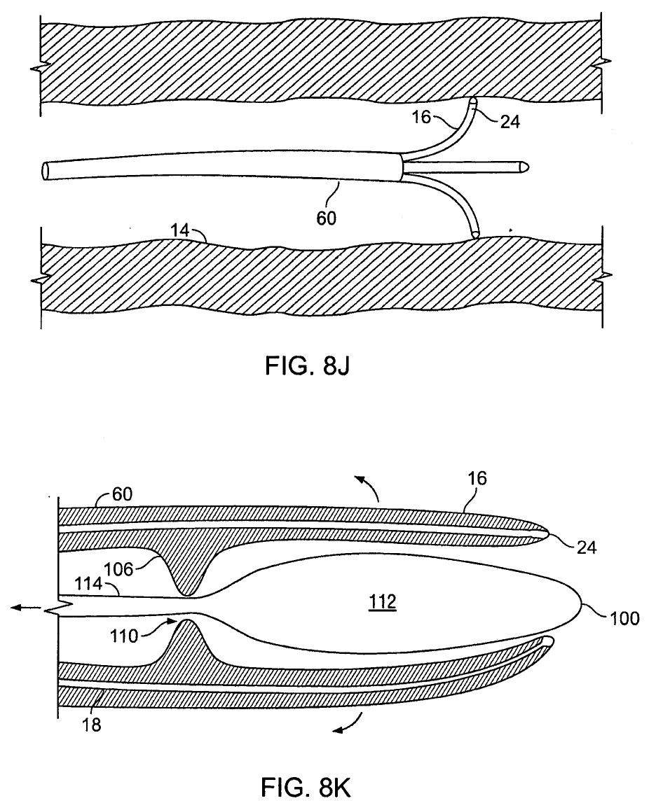

In a seventh embodiment, shown in FIGS. 8A-B, a plurality of probes 16 passes through a cannula 60. The distal ends of the probes 16 are attached to anchor points circumferentially distributed around a hub 78. The hub 78 is coupled to a control wire 80 that enables it to be moved along the longitudinal axis of the cannula 60 to either deploy the probes 16 (FIG. 8A) or to retract the probes 16 (FIG. 8B). However, in other embodiments, the hub 78 remains stationary and it is the cannula 60 that is moved proximally and distally to either deploy or recover the probes 16.

The probes 16 are pre-formed to bow outward as shown in FIG. 8A so as to contact the arterial wall 14 at an intermediate point between the hub 78 and the cannula 60. Optional spacer rings 64, like those discussed in connection with FIGS. 6A-C, are attached to the probes 16 at one or more points along their distal sections to maintain their relative positions. In this seventh embodiment, the atraumatic light-coupler 24 includes a side-window 82 located at the intermediate point. The side window 82 faces radially outward so that when the probe 16 is fully deployed, the side window 82 atraumatically contacts the arterial wall 14.

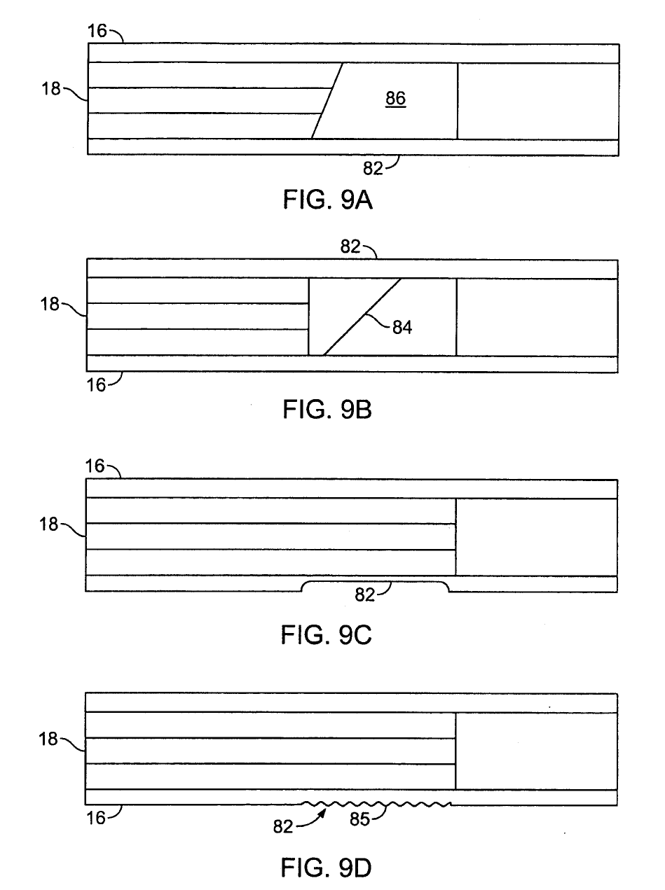

An atraumatic light-coupler 24 for placement along the side of the probe 16 includes a right-angle reflector 84, such as a prism or mirror, placed in optical communication between the fiber 18 and the side window 82, as shown in FIG. 9B. Alternatively, an air gap 86 is placed in optical communication between the tip of an angle polished fiber 18 and the side-window 82, as shown in FIG. 9A.

FIGS. 9C-9D shows additional examples of atraumatic light-couplers 24 for placement along the side of the probe 16. In these examples, the side window 82 is formed by a portion of the fiber's cladding that is thin enough to allow passage of light. The side window 82 can be left exposed, as shown in FIG. 9C, or a diffraction grating 85 can be placed in optical communication with the side window 82 to further control the direction of the beam, as shown in FIG. 9C.

When the hub 78 and the cannula 60 are drawn together, as shown in FIG. 8B, they can easily be guided to a location of interest. Once the hub 78 and cannula 60 reach a location of interest, one either advances the hub 78 or retracts the cannula 60. In either case, the probes 16 are released from the confines of the cannula 60, as shown in FIG. 8A. Once free of the radially restraining force applied by the cannula's inner wall, the probes 16 assume their natural shape, bowing outward, as shown in FIG. 8B, so that their respective side-windows 82 atraumatically contact the arterial wall 14. The atraumatic light-couplers 24 guide light from the light source 50 through the side windows 82. At the same time, the atraumatic light-couplers 24 recover re-emergent light from the wall 14 through the side windows 82 and pass it into the fibers 16, which guide that light to the photo-detector 52.

When the examination of the wall 14 is complete, the hub 78 and cannula 60 are brought back together, as shown in FIG. 8B, and the probes 16 are once again confined inside the cannula 60.

In an eighth embodiment, shown in FIGS. 8C-D, the cannula 60 has a proximal section 88 and a distal section 90 separated by a circumferential gap 92, as described in connection with the third embodiment (FIGS. 5D-F) and the fifth embodiment (FIGS. 6D-F). Unlike the third and fifth embodiments, in which the distal tips of the probes 16 atraumatically contact the wall 14, in the eighth embodiment the distal tips of the probes 16 are attached to a hub 78 at the distal section 90 of the cannula 60. Like the probes 16 of the seventh embodiment, the probes 16 of the eighth embodiment have side windows 82 at intermediate points for atraumatically contacting the arterial wall 14. An actuator (not shown) is mechanically coupled to selectively apply tension to the probes 16. When the probes 16 are under tension, they lie against the distal section 90 of the cannula 60, as shown in FIG. 8D. When probes 16 are relaxed, they spring radially outward, away from the distal section 90, enough so that the side windows 82 at the intermediate sections atraumatically contact the arterial wall 14.

In use, the cannula 60 is guided to a region of interest with the probes 16 placed under tension. The probes 16 are thus drawn against the cannula 60, as shown in FIG. 8B. Once at the region of interest, the tension is released, and the probes 16 spring radially outward, as shown in FIG. 8A, so that the side windows 82 atraumatically contact the wall 14. After data collection, the probes 16 are again placed under tension to draw them back against the cannula 60, as shown in FIG. 8B.

In the seventh and eighth embodiments, a particular probe 16 emerges from the cannula 60 at an exit point and re-attaches to the hub 78 at an anchor point. In a cylindrical coordinate system centered on the axis of the cannula 60, the exit point and the anchor point have different axial coordinates but the same angular coordinate. However, as FIGS. 8E and 8F illustrate, this need not be the case.

FIG. 8E shows a ninth embodiment in which a cannula 60 has a plurality of exit holes 96 and a corresponding plurality of entry holes 98. Each probe 16 exits the cannula 60 through an exit hole 96 and re-enters the cannula 60 through an entry hole 96 that is circumferentially offset from its corresponding exit hole. This results in the helical arrangement shown in FIG. 8E. The extent of the circumferential offset defines the pitch of the helix.

The distal ends of the probe 16 are attached to a hub 78 (not shown) inside the cannula 60. Each probe 16 has a side window 82 between the exit hole and the corresponding entry hole. A control wire 80 within the cannula 60 (not shown) deploys the probes 16, as shown, or retracts them so that they rest against the exterior of the cannula 60. A guide-wire 63 passing through the cannula 60 and exiting out the distal tip thereof enables the cannula 60 to be guided to a region of interest.

FIG. 8F shows a tenth embodiment in which a cannula 60 has a distal section 88 and a proximal section 90. The proximal and distal sections of the cannula 60 surround a central shaft 100 having an exposed portion 102. Probes 16 extend axially through a gap between the shaft and the cannula 60. The probes 16 are anchored at their distal ends at circumferentially displaced anchor points on a hub 78 attached to the shaft 100. The circumferential offset causes the helical configuration of the probes 16 in FIG. 8F. The extent of this circumferential offset defines a pitch of the helix.

An actuator (not shown) selectively applies tension to the probes 16. When the probes 16 are under tension, they retract against the exposed portion 102 of the central shaft 100. When the probes 16 are relaxed, they assume the configuration shown in FIG. 8F, in which they spring radially outward from the exposed portion 102 of the central shaft 100 so that their side windows 82 atraumatically contact the arterial wall 14.

In the embodiments described thus far, the probes 16 and the cannula 60 have been separate structures. However, the probes 16 can also be integrated, or otherwise embedded in the cannula 60. In this case, portions of the cannula 60 extend radially outward to contact the arterial wall 14.

FIGS. 8G and 8H show an eleventh embodiment in a deployed and retracted state, respectively. The eleventh embodiment includes slots 104 cut into the wall of the cannula 60 enclosing an internal shaft 100. Pairs of adjacent slots 104 define probe portions 16 of the cannula 60. The probe portions 16 buckle outward when the distal tip of the cannula 60 is pulled proximally, as shown in FIG. 8G. When the distal tip of the cannula 60 is extended, the probe portions 16 lay flat against the shaft 100, as shown in FIG. 8H.

Each probe portion 16 has a side window 82 for atraumatically contacting the wall 14 when the probe portion 16 is deployed. The side window 82 is in optical communication with an atraumatic coupler 24. An optical fiber embedded within the wall of the cannula 60 provides an optical path to and from the atraumatic coupler 24.

FIGS. 8I-J show a twelfth embodiment in a deployed and retracted state. The twelfth embodiment includes slots 104 cut into the wall of the cannula 60 enclosing an internal shaft 100. Unlike the slots 104 in the eleventh embodiment, the slots 104 in the twelfth embodiment extend all the way to the distal tip of the cannula. Pairs of adjacent slots 104 define probe portions 16 of the cannula 60.

As shown in the cross-section of FIG. 8K, the cannula 60 includes radially-inward projections 106 forming a throat 110. The shaft 100 has a bulbous portion 112 distal to the throat 110 and a straight portion 114 extending proximally through the throat 110 to join the bulbous portion 112. The probe portions 16 are biased to rest against the bulbous portion 112 of the shaft 100, as shown in FIG. 8I. When the shaft 100 is drawn proximally, the bulbous portion 112 wedges against the projections 106. This forces the probe-portions 16 to pivot radially outward, as shown in FIG. 8J.

Each probe portion 16 has an atraumatic coupler 24 at its distal tip for atraumatically contacting the wall 14 when the probe portion 16 is deployed. An optical fiber embedded within the wall of the cannula 60 provides an optical path to and from the atraumatic coupler 24.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-32. (canceled)

33. An apparatus for detecting vulnerable plaque within a lumen defined by an intraluminal wall, the apparatus comprising:
a cannula having a longitudinal axis;
a plurality of probes extending through the cannula, each probe having
an optical fiber extending therethrough, and
an atraumatic light-coupler in optical communication with the optical fiber, the coupler being configured to atraumatically contact the intraluminal wall.

34. The apparatus of claim 33, further comprising a spacer ring attached to each of the probes for maintaining the positions of the probes relative to each other.

35. The apparatus of claim 33, further comprising a hub attached to a distal end of each of the probes.

36. The apparatus of claim 35, wherein the distal end of the probe is attached to the hub at an anchor point that is circumferentially offset from a proximal portion of the probe.

37. The apparatus of claim 35, further comprising a spacer ring attached to each of the probes for maintaining the positions of the probes relative to each other.

38. The apparatus of claim 35, wherein each of the probes resiliently assumes a bow shape having a point of inflection between the hub and the cannula.

39. The apparatus of claim 33, wherein each of the probes resiliently assumes a desired shape.

40. The apparatus of claim 33, wherein the atraumatic coupler comprises means for providing optical communication between the optical fiber and the intraluminal wall.

41. The apparatus of claim 33, wherein at least one of the plurality of probes is integral with the cannula.

42. The apparatus of claim 33, wherein the optical fiber is embedded within the cannula.

43-55. (canceled)

56. The apparatus of claim 36, further comprising a control wire, wherein the control wire passes through the cannula, is coupled to the hub, and is operable to deploy the probes or undeploy the probes.

57. The apparatus of claim 33, further comprising a guidewire lumen within the cannula, said lumen configured to accommodate a guidewire for enabling the cannula to be guided to a region of interest.

58. The apparatus of claim 33, further comprising:
a light source in optical communication with the optical fibers; and
a detector in optical communication with at least one of the optical fibers, said detector configured to detect a light signal of a corresponding atraumatic light coupler.

59. An apparatus for detecting vulnerable plaque within a lumen defined by an intraluminal wall, the apparatus comprising:
a cannula having a longitudinal axis;
a plurality of probes extending through the cannula, each probe having
an optical fiber extending therethrough, and
an atraumatic light-coupler in optical communication with the optical fiber, the
coupler being configured to atraumatically contact the intraluminal wall; and
a hub attached to a distal end of each of the probes;
wherein, in a deployed state, each of the probes resiliently assumes a bow shape having a point of inflection between the hub and the cannula.

60. The apparatus of claim 59, further comprising a control wire, wherein the control wire passes through the cannula, is coupled to the hub, and is operable to deploy the probes or undeploy the probes.

61. The apparatus of claim 59, wherein the plurality of probes comprises six or more.

62. The apparatus of claim 59, wherein each atraumatic light-coupler includes a side window disposed to face radially outward such that the side window is enabled to contact the intraluminal wall.

63. The apparatus of claim 59, further comprising a guidewire lumen within the cannula, said lumen configured to accommodate a guidewire for enabling the cannula to be guided to a region of interest.

64. A method for analyzing a lumen, said method comprising:
deploying each of a plurality of atraumatic light couplers along discrete trajectories to contact a wall of the lumen;
illuminating each contact point by way of the corresponding atraumatic light coupler;
receiving a light signal from each atraumatic light coupler; and
analyzing each light signal to identify a property of the lumen.

65. The method of claim 64, wherein the lumen is a blood vessel and the analyzing step includes analyzing one or more of the light signals to identify the presence of a vulnerable plaque.

66. The method of claim 65, further comprising inserting into the blood vessel a cannula containing the atraumatic couplers, said inserting step being performed while the atraumatic couplers are constrained towards a longitudinal axis of the cannula;
wherein the deploying step occurs after the inserting step and includes deploying the atraumatic couplers radially outward from the longitudinal axis.

67. An intraluminal apparatus comprising:
a cannula having a longitudinal axis; and
a plurality of probes extending through the cannula, each probe having
an optical fiber extending therethrough, and
an atraumatic light-coupler in optical communication with the optical fiber;
wherein, at a distal end of the cannula, each probe is deployable away from the longitudinal axis, along a trajectory distinct from each other probe, such that the couplers atraumatically contact an intraluminal wall.

68. The apparatus of claim 67, wherein the cannula is sized and configured for intravascular diagnosis of a blood vessel wall.

69. The apparatus of claim 68, wherein the blood vessel wall is a human coronary artery wall.

70. A conformable multi-arm optical catheter, comprising:
a proximal end;
a distal end;
a central axis;
a proximal catheter segment;
a distal interrogation section extending from the distal end of the proximal catheter segment, wherein the interrogation section comprises at least two flexible probe arms that in an unconstrained state radially bow out from the central axis and then, proceeding distally, bow back toward the central axis of the catheter; and
a distal insertion segment connected to the distal ends of the probe arms and providing a guidewire lumen so that the distal insertion segment is slideably engageable with the guidewire,
wherein each probe arm comprises at least one optical fiber entering the probe arm and terminating at or near the most radially extendable portion of the probe arm in a side-viewing configuration or assembly to form a side-viewing optical probe element capable of transmitting and collecting light.
